(19) United States
(12) Patent Application Publication
(10) Pub. No.: US 2023/0355591 A1
(43) Pub. Date: Nov. 9, 2023

(54) Pharmaceutical composition and use thereof

(71) Applicant: ALPHALA CO., LTD., Taipei City (TW)

(72) Inventors: Chi-Jen HONG, Taipei City (TW); Yen-Fu CHEN, Taipei City (TW); Cheng-Ho CHUNG, Taipei City (TW)

(21) Appl. No.: 17/312,557

(22) PCT Filed: Jan. 8, 2021
(86) PCT No.: PCT/US2021/012584

§ 371 Date: Jun. 10, 2021

Related U.S. Application Data
(60) Provisional application No. 62/959,205, filed on Jan. 10, 2020.

(51) Int. Cl. A61K 31/4245; A61K 9/16; A61K 9/28; A61K 9/20

(52) U.S. Cl. CPC A61K 31/4245; A61K 9/1641; A61K 9/1652; A61K 9/2886; A61K 9/2013; A61K 9/2054; A61K 9/2009; A61K 9/2866; A61K 9/2813; A61K 9/282; A61K 9/2853

(57) ABSTRACT

An oral pharmaceutical formulation containing an effective amount of NRC-AN-019 including its pharmaceutically acceptable salts and polymorphs thereof, by dispersing in a polymer system in a final state of subdivision to enhance oral bioavailability. It also compositions for the treatment of Chronic Myeloid Leukemia and other tumors such as head and neck cancer, prostate cancer and the like.

This application claims the benefit of filing date of U.S. Provisional Application Ser. No. 62/959,205, filed Jan. 10, 2020, which is hereby incorporated by reference.

### FIELD OF THE DISCLOSURE

The present disclosure relates to a pharmaceutical composition comprising a glucagon receptor antagonist, and the use thereof.

### BACKGROUND

ALP001E is a glucagon receptor antagonist and used for treatment of diabetes mellitus. ALP001E is a Biopharmaceutical Classification System (BCS) Class II drug which indicates poor aqueous solubility but good membrane permeability. It is stable in the solid state and not sensitive to light irradiation. However, ALP001E predominantly undergoes degradation by hydrolytic pathways in the presence of moisture. It is also sensitive to acid or basic environments.

Because of these physicochemical and biopharmaceutical properties of ALP001E, several attempts have been made to provide compositions of ALP001E that are stable and provide desirable in vitro release and bioavailability.

Thus, it is desirable to provide a novel pharmaceutical composition comprising ALP01E, so that ALP001E can be applied to clinical use.

### SUMMARY

The present disclosure relates to a novel pharmaceutical composition comprising a glucagon receptor antagonist, ALP001E, with good stability and desired in vitro or in vivo performance.

An aspect of the present disclosure is drawn to a pharmaceutical composition, comprising: an active ingredient of the following formula (I) or a pharmaceutically acceptable salt or solvate thereof:

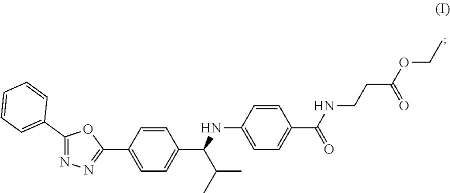 [SMILES: CCOC(=O)CCNC(=O)c1ccc(N[C@H](c2ccc(-c3nnc(-c4ccccc4)o3)cc2)C(C)C)cc1]

one or more excipient selected from the group consisting of a binder, a diluent, a disintegrant, a dispersant, an emulsifier, a filler, a glidant, a lubricant, a plasticizer, a solubilizer, a thickener and a wetting agent.

In addition to the active ingredient of the formula (I) described above, their pharmaceutically acceptable salts and solvates, where applicable, are also covered by the present disclosure. A salt can be formed between an anion and a positively charged group (e.g., amino) on a compound. Examples of a suitable anion include chloride, bromide, iodide, sulfate, nitrate, phosphate, citrate, methanesulfonate, trifluoroacetate, acetate, malate, tosylate, tartrate, fumurate, glutamate, glucuronate, lactate, glutarate, and naleate. A salt can also be formed between a cation and a negatively charged group. Examples of a suitable cation include sodium ion, potassium ion, magnesium ion, calcium ion, and an ammonium cation such as tetramethylammonium ion. A salt further includes those containing quaternary nitrogen atoms. A solvate refers to a complex formed between an active compound and a pharmaceutically acceptable solvent. Examples of a pharmaceutically acceptable solvent include water, ethanol, isopropanol, ethyl acetate, acetic acid, and ethanolamine.

Another aspect of the present disclosure is drawn to a method for reducing the glycemic level in a subject, comprising administering to the subject in need thereof the aforesaid pharmaceutical composition.

A further aspect of the present disclosure is drawn to a method of treating disorders associated with glucagon, comprising administering to the subject in need thereof the aforesaid pharmaceutical composition.

The pharmaceutical composition of the present invention can be administered to a subject orally, parenterally, by inhalation spray, topically, rectally, nasally, buccally, or via an implanted reservoir. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques.

In the present disclosure, the pharmaceutical composition can be an oral pharmaceutical composition, which can be any orally acceptable dosage form including capsules, tablets, emulsions and aqueous suspensions, dispersions, and solutions. Oral solid dosage forms can be prepared by spray dried techniques; hot melt extrusion strategy, micronization, and nano milling technologies. Alternatively, the pharmaceutical composition can be a nasal aerosol or inhalation composition, which can be prepared according to techniques well known in the art of pharmaceutical formulation. Further alternatively, the pharmaceutical composition of the present disclosure can also be administered in the form of suppositories for rectal administration.

The excipient in the pharmaceutical composition must be “acceptable” in the sense that it is compatible with the active ingredient of the composition (and preferably, capable of stabilizing the active ingredient) and not deleterious to the subject to be treated.

The term “treating”, “treat” or “treatment” refers to application or administration of the active ingredient to a subject with the purpose to cure, alleviate, relieve, alter, remedy, improve, or affect the disease, the symptom, or the predisposition. “An effective amount” refers to the amount of the active ingredient which is required to confer the desired effect on the subject. Effective amounts vary, as recognized by those skilled in the art, depending on route of administration, excipient usage, and the possibility of co-usage with other therapeutic treatments such as use of other active agents.

The term “weight percentages” (i.e., “% by weight” and “wt %” and w/w) referenced herein, unless otherwise indicated, are based on the total weight of the pharmaceutical composition unless specified otherwise.

The details of one or more embodiments of the disclosure are set forth in the description below. Other features, objects, and advantages of the disclosure will be apparent from the description and from the claims.

### DETAILED DESCRIPTION

Disclosed in detail below is a pharmaceutical composition, comprising: an active ingredient of the following formula (I) or a pharmaceutically acceptable salt or solvate thereof:

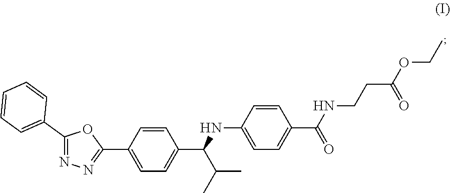 [SMILES: CCOC(=O)CCNC(=O)c1ccc(N[C@H](c2ccc(-c3nnc(-c4ccccc4)o3)cc2)C(C)C)cc1]

one or more excipient selected from the group consisting of a binder, a diluent, a disintegrant, a dispersant, an emulsifier, a filler, a glidant, a lubricant, a plasticizer, a solubilizer, a thickener and a wetting agent.

The present disclosure provides a novel pharmaceutical composition of ALP001E, which may optionally comprise at least one pharmaceutically acceptable excipient. The pharmaceutical composition provided by the present disclosure is chemically and polymorphically stable. In addition, the pharmaceutical composition of the present disclosure exhibits desired in vitro and in vivo performance and can be prepared by simple, ono-tedious and cost-effective process, such as spray dry method. Furthermore, the dry powders of the pharmaceutical composition provided by the present disclosure have acceptable solubility and increased oral absorption by a specific formulation ratio and process parameters, which can carry out the possibility of entering the GMP pharmaceutical industry.

Examples of the binder may include celluloses (such as ethyl cellulose, cellulose acetate, carboxymethyl cellulose calcium, sodium carboxymethyl cellulose, methyl cellulose, hydroxyethylcellulose (HEC), hydroxypropylcellulose (HPC), hydroxypropyl methyl cellulose (HPMC)), cross-linked polymers (such as soluplus), gelatin, microcrystalline celluloses, natural and synthetic gums (such as acacia, alginic acid, alginates, extract of Irish moss, Panwar gum, ghatti gum, mucilage of isabgol husks, carboxymethylcellulose, methylcellulose, polyvinylpyrrolidone (PVP), Veegum, larch arabogalactan, powdered tragacanth, and guar gum), starches (such as corn starch, potato starch, pre-gelatinized starch and partially pre-gelatinized maize starch), sugars (such as sucrose, glucose, dextrose, molasses, and lactose) or a combination thereof; but the present disclosure is not limited thereto.

Examples of the diluent may include calcium sulfate, cellulose, dibasic calcium phosphate, dry starch, inositol, kaolin, lactose, mannitol, microcrystalline cellulose, powdered sugar, sodium chloride, sorbitol, starch (such as directly compressed starch and hydrolyzed starch), sucrose, or a combination thereof; but the present disclosure is not limited thereto.

Examples of the disintegrant may include agar, alginic acid, alginate, aligns, bentonite, calcium carbonate, cation-exchange resins, celluloses (such as methylcellulose and carboxymethylcellulose), citrus pulp, clays, cross-linked celluloses (such as croscarmellose and croscarmellose sodium), cross-linked polymers (such as crospovidone), cross-linked starches, gums (such as guar gum and Veegum HV), microcrystalline cellulose (such as various types of sodium starch glycolate), natural sponge, polacrilin potassium, starches (such as corn starch, potato starch, tapioca starch, and pre-gelatinized starch), wood products or a combination thereof; but the present disclosure is not limited thereto.

Examples of the dispersant may include acacia, hydroxypropyl methylcellulose, hypromellose acetate succinate (HPMCAS), pectin, polyvinylpyrolidone, sodium carbomethylcellulose, sodium carboxymethylcellulose, tragacanth, Veegum or a combination thereof; but the present disclosure is not limited thereto.

Examples of the emulsifier may include acacia, bentonite, gelatin, tragacanth, triethanolamine oleate, Tween 20, Tween 80 or a combination thereof; but the present disclosure is not limited thereto.

Examples of the filler may include calcium carbonate, dextrates, kaolin, mannitol, microcrystalline cellulose, pre-gelatinized starch, powdered cellulose, silicic acid, sorbitol, starch, tale or a combination thereof; but the present disclosure is not limited thereto.

Examples of the glidant (also named as the flowing agent) may include corn starch, tale, solicon dioxide, colloidal silicone dioxide, or a combination thereof; but the present disclosure is not limited thereto.

Examples of the lubricant may include agar, calcium stearate, ethyl laureate, ethyl oleate, glycerin, glycols (such as glycerol behenate and polyethylene glycol (PEG)), hydrogenated vegetable oil (such as peanut oil, cottonseed oil, sunflower oil, sesame oil, olive oil, corn oil, and soybean oil), light mineral oil, lycopodium, magnesium stearate, mannitol, mineral oil, silica or silica gels, sodium lauryl sulfate, sodium stearyl fumarate, sorbitol, stearic acid, tale, wax, zinc stearate or a combination thereof; but the present disclosure is not limited thereto.

Examples of the plasticizer may include castor oil, citrate esters (such as triethyl citrate, tributyl citrate, acetyl triethyl citrate and acetyl tributyl citrate), fatty acid esters (such as butyl stearate, glycerol monostearate and stearyl alcohol), glycol derivatives (such as polyethylene glycol and propylene glycol), mineral oil, phthalate esters (such as diethyl phthalate, dibutyl phthalate and dioctyl phosphate), sebacate esters (such as dibutyl sebacate), triacetin, vitamin E TPGS (D-α-tocopheryl polyethylene glycol 1000 succinate) or a combination thereof; but the present disclosure is not limited thereto.

Examples of the solubilizer may include citric acid, tartaric acid, benzoic acid, polyethylene glycol, ethanol, propylene glycol, glycerin, n-methyl 2-pyrrolidone, dimethyl acetamide, beeswax, d-α tocopherol, oleic acid, mono and di glycerides, cremphor, Tween 20, sorbitan monooleate, peppermint oil, polysorbate, peanut oil, corn oil, soybean oil, sesame oil, olive oil, cotton seed oil, α-cyclodextrins, β-cyclodextrins, γ-cyclodextrins, sulfobutylether-cyclodextrin, hydroxypropyl-cyclodextrin, glycerol, choline, distearoyl phosphatidylglycerol (DSPG), dimyristoylphosphatidylcholine (DMPC), dimyristoylphosphatidylglycerol (DMPG), Macrogol 15 Hydroxystearate or a combination thereof; but the present disclosure is not limited thereto.

Examples of the thickener may include acacia, agar, alamic acid, alginic acid, aluminum monostearate, attapulgite, bentonite, carbomer, carbomer copolymer, carbomer homopolymer, carbomer interpolymer, carboxymethylcellulose calcium, carboxymethylcellulose sodium, carrageenan, cellulose, dextrin, gelatin, gum (such as gellan gum, guar gum and xanthan gum), hydroxyethyl cellulose, hydroxypropyl cellulose, hypromellose, magnesium aluminum silicate, maltodextrin, methylcellulose, microcrystalline cellulose, pectin, polyethylene oxide, polyvinyl alcohol, povidone, propylene glycol aliginate, silicon dioxide, colloidal silicon dioxide, sodium alginate, starch (including corn starch, potato starch, tapioca starch and wheat starch), tragacanth or a combination thereof; but the present disclosure is not limited thereto.

Examples of the wetting agent may include diethylene glycol monolaurate, propylene glycol monostearate, polyoxyethyiene lauryl ether, sorbitan monooleate, sodium lauryl sulfate (SLS), sodium stearyl fumarate (SSF), Tween 20, Tween 80 or a combination thereof; but the present disclosure is not limited thereto.

In one embodiment of the present disclosure, a content of the active ingredient may be ranged from 5 wt % to 50 wt % based on a total weight of the pharmaceutical compostion. For example, the content may be ranged from 5 wt % to 45 wt %, 5 wt % to 40 wt %, 8 wt % to 40 wt %, 8 wt % to 35 wt %, 9 wt % to 35 wt %, 10 wt % to 35 wt %, 10 wt % to 32 wt %, 11 wt % to 32 wt %, 12 wt % to 32 wt %. 12 wt % to 30 wt %, 12 wt % to 27 wt %, 12 wt % to 26 wt %, 13 wt % to 26 wt %, 13 wt % to 25 wt %, 14 wt % to 25 wt %, 14 wt % to 24 wt %, 15 wt % to 24 wt %, 15 wt % to 23 wt %, 16 wt % to 23 wt %, 16 wt % to 22 wt %, 17 wt % to 22 wt %, 17 wt % to 21 wt %. 17 wt % to 20 wt %, 17 wt % to 19 wt % or 18 wt % to 19 wt % based on the total weight of the pharmaceutical composition.

In another embodiment of the present disclosure, the excipient may comprise the dispersant and the solubilizer.

In the aforesaid embodiment, the dispersant may be hypromellose acetate succinate.

In the aforesaid embodiments, the solubilizer may be Macrogol 15 Hydroxystearate.

In the aforesaid embodiments, a content of the dispersant may be ranged from 40 wt % to 90 wt % based on a total weight of the pharmaceutical composition. For example, the content may be ranged from 45 wt % to 90 wt %, 50 wt % to 90 wt %, 55 wt % to 90 wt %, 55 wt % to 85 wt %, 55 wt % to 80 wt %, 60 wt % to 80 wt %, 60 wt % to 75 wt %, 65 wt % to 75 wt % or 70 wt % to 75 wt % based on the total weight of the pharmaceutical composition.

In the aforesaid embodiments, a content of the solubilizer may be ranged from 3 wt % to 20 wt % based on a total weight of the pharmaceutical composition. For example, the content may be ranged from 4 wt % to 20 wt %, 4 wt % to 19 wt %, 5 wt % to 19 wt %, 5 wt % to 18 wt %, 6 wt % to 18 wt %, 6 wt % to 17 wt %, 6 wt % to 16 wt %, 7 wt % to 16 wt %, 7 wt % to 15 wt %, 7 wt % to 14 wt %, 8 wt % to 14 wt %, 8 wt % to 13 wt %, 8 wt % to 12 wt %, 8 wt % to 11 wt %, 8 wt % to 10 wt % or 8 wt % to 9 wt % based on the total weight of the pharmaceutical composition.

In the aforesaid embodiments, a weight ratio of the dispersant to the solubilizer can be ranged from 3.0 to 20.0. For example, a weight ratio of hypromellose acetate succinate to Macrogol 15 Hydroxystearate can be ranged from 4.0 to 19.6, 4.0 to 15.0, 4.0 to 13.0, 5.0 to 13.0, 5.0 to 10.0, 6.0 to 10.0, 6.0 to 9.5, 7.0 to 9.5, 7.5 to 9.5, 8.0 to 9.5, 8.5 to 9.5 or 8.5 to 9.3. When this weight ratio is in the aforesaid range, the pharmaceutical composition of the present disclosure have an enhance effect for improving of solubility or oral bioavailability in vivo.

In another embodiment of the present disclosure, the excipient may comprise the binder, the disintegrant, the dispersant, a diluent, the glidant, the lubricant and the plasticizer.

In the aforesaid embodiments, the binder may be microcrystalline cellulose.

In the aforesaid embodiments, the disintegrant may be croscarmellose sodium.

In the aforesaid embodiments, the dispersant may be hypromellose acetate succinate.

In the aforesaid embodiments, the diluent may be dibasic calcium phosphate.

In the aforesaid embodiments, the glidant may be silicon dioxide.

In the aforesaid embodiments, the lubricant may be sodium stearyl fumarate.

In the aforesaid embodiments, the plasticizer may be dibutyl sebacate.

In the aforesaid embodiment, a content of the binder may be ranged from 15 wt % to 50 wt % based on a total weight of the pharmaceutical composition. For example, the content may be ranged from 20 wt % to 50 wt %, 20 wt % to 45 wt %, 25 wt % to 45 wt %, 25 wt % to 40 wt %, 30 wt % to 40 wt % or 30 wt % to 35 wt % based on a total weight of the pharmaceutical composition.

In the aforesaid embodiments, a content of the disintegrant may be ranged from 0.5 wt % to 10 wt % based on a total weight of the pharmaceutical composition. For example, the content may be ranged from 0.5 wt % to 8 wt %, 1 wt % to 8 wt %, 1 wt % to 7 wt %, 1 wt % to 6 wt %, 1 wt % to 5 wt %, 1.5 wt % to 5 wt %, 1.5 wt % to 4 wt %, 2 wt % to 4 wt %, 2 wt % to 3.5 wt % or 2.5 wt % to 3.5 wt % based on a total weight of the pharmaceutical composition.

In the aforesaid embodiments, a content of the dispersant may be ranged from 15 wt % to 50 wt % based on a total weight of the pharmaceutical composition. For example, the content may be ranged from 15 wt % to 45 wt %, 18 wt % to 45 wt %, 18 wt % to 40 wt %, 20 wt % to 40 wt %, 20 wt % to 35 wt %, 23 wt % to 35 wt %, 23 wt % to 33 wt %, 25 wt % to 33 wt % or 27 wt % to 33 wt % based on a total weight of the pharmaceutical composition.

In the aforesaid embodiments, a content of the diluent may be ranged from 10 wt % to 30 wt % based on a total weight of the pharmaceutical composition. For example, the content may be ranged from 10 wt % to 25 wt %, 12 wt % to 25 wt %, 12 wt % to 23 wt %, 14 wt % to 23 wt %, 14 wt % to 20 wt % or 14 wt % to 18 wt % based on a total weight of the pharmaceutical composition.

In the aforesaid embodiments, a content of the glidant may be ranged from 0.5 wt % to 10 wt % based on a total weight of the pharmaceutical composition. For example, the content may be range from 0.5 wt % to 8 wt %, 0.5 wt % to 6 wt %, 0.5 wt % to 4 wt %, 1 wt % to 4 wt %, 1 wt % to 3 wt %, 1.5 wt % to 3 wt % or 1.5 wt % to 2.5 wt % based on a total weight of the pharmaceutical composition.

In the aforesaid embodiments, a content of the lubricant may be ranged from 0.5 wt % to 10 wt % based on a total weight of the pharmaceutical composition. For example, the content may be ranged from 0.5 wt % to 8 wt %, 0.5 wt % to 6 wt %, 0.5 wt % to 4 wt %, 1 wt % to 4 wt %, 1 wt % to 3 wt %, 1 wt % to 2.5 wt % or 1 wt % to 2 wt % based on a total weight of the pharmaceutical composition.

In the aforesaid embodiments, a content of the plasticizer may be ranged from 0.5 wt % to 10 wt % based on a total weight of the pharmaceutical composition. For example, the content may be ranged from 0.5 wt % to 8 wt %, 1 wt % to 8 wt %, 1 wt % to 7 wt %, 1 wt % to 6 wt %, 1 wt % to 5 wt %, 1.5 wt % to 5 wt %, 1.5 wt % to 4 wt %, 2 wt % to 4 wt %, 2 wt % to 3.5 wt % or 3 wt % to 3.5 wt % based on a total weight of the pharmaceutical composition.

In another embodiment of the present disclosure, the excipient may comprise the binder, the dispersant, the diluent, the glidant, the lubricant and the solubilizer.

In the aforesaid embodiments, the binder may be metolose.

In the aforesaid embodiments, the dispersant may be hypromellose acetate succinate.

In the aforesaid embodiments, the diluent may be dibasic calcium phosphate.

In the aforesaid embodiments, the glidant may be silicon dioxide.

In the aforesaid embodiments, the lubricant may be sodium stearyl fumarate.

In the aforesaid embodiments, the solubilizer may be Macrogol 15 Hydroxystearate.

In the aforesaid embodiments, a content of the binder may be ranged from 15 wt % to 50 wt % based on a total weight of the pharmaceutical composition. For example, the content may be ranged from 20 wt % to 50 wt %, 20 wt % to 45 wt %, 25 wt % to 45 wt %, 25 wt % to 40 wt %, 30 wt % to 40 wt % or 30 wt % to 35 wt % based on a total weight of the pharmaceutical composition.

In the aforesaid embodiments, a content of the the dispersant may be ranged from 20 wt % to 60 wt % based on a total weight of the pharmaceutical composition. For example, the content may be ranged from 20 wt % to 55 wt %, 25 wt % to 55 wt %, 25 wt % to 50 wt %, 30 wt % to 50 wt %, 30 wt % to 45 wt %, 35 wt % to 45 wt %, 35 wt % to 43 wt % or 38 wt % to 43 wt % based on a total weight of the pharmaceutical composition.

In the aforesaid embodiments, a content of the diluent may be ranged from 5 wt % to 20 wt % based on a total weight of the pharmaceutical composition. For example, the content may be ranged from 5 wt % to 18 wt %, 6 wt % to 18 wt %, 6 wt % to 15 wt %, 7 wt % to 15 wt %, 7 wt % to 13 wt %, 8 wt % to 13 wt % or 8 wt % to 12 wt % based on a total weight of the pharmaceutical composition.

In the aforesaid embodiments, a content of the glidant may be ranged from 0.5 wt % to 10 wt % based on a total weight of the pharmaceutical composition. For example, the content may be range from 0.5 wt % to 8 wt %, 0.5 wt % to 6 wt %, 0.5 wt % to 4 wt %, 1 wt % to 4 wt %, 1 wt % to 3 wt %, 1.5 wt % to 3 wt % or 1.5 wt % to 2.5 wt % based on a total weight of the pharmaceutical composition.

In the aforesaid embodiments, a content of the the lubricant may be ranged from 0.5 wt % to 10 wt % based on a total weight of the pharmaceutical composition. For example, the content may be ranged from 0.5 wt % to 8 wt %, 0.5 wt % to 6 wt %, 0.5 wt % to 4 wt %, 1 wt % to 4 wt %, 1 wt % to 3 wt %, 1 wt % to 2.5 wt % or 1 wt % to 2 wt % based on a total weight of the pharmaceutical composition.

In the aforesaid embodiments, a content of the the solubilizer may be ranged from 1 wt % to 10 wt % based on a total weight of the pharmaceutical composition. For example, the content may be ranged from 1 wt % to 8 wt %, 2 wt % to 8 wt %, 2 wt % to 7 wt %, 3 wt % to 7 wt %, 3 wt % to 6 wt %, 4 wt % to 6 wt % or 4 wt % to 5 wt % based on a total weight of the pharmaceutical composition.

In another embodiment of the present disclosure, the excipient may comprise the binder and the solubilizer.

In the aforesaid embodiments, the binder may be Eudragit FS100, soluplus or a combination thereof.

In the aforesaid embodiments, the solubilizer may be Macrogol 15 Hydroxystearate.

In the aforesaid embodiments, a content of the binder may be ranged from 40 wt % to 80 wt % based on a total weight of the pharmaceutical composition. For example, the content may be ranged from 45 wt % to 80 wt %, 50 wt % to 80 wt %, 55 wt % to 80 wt %, 60 wt % to 80 wt %, 65 wt % to 80 wt %, 65 wt % to 75 wt % or 70 wt % to 75 wt % based on a total weight of the pharmaceutical composition.

In the aforesaid embodiments, a content of the solibilizer may be 5 wt % to 15 wt % based on a total weight of the pharmaceutical composition. For example, the content may be ranged from 5 wt % to 13 wt %, 6 wt % to 13 wt %, 6 wt % to 12 wt %, 7 wt % to 12 wt %, 7 wt % to 11 wt %, 7 wt % to 10 wt %, 7 wt % to 9 wt % or 8 wt % to 9 wt % based on a total weight of the pharmaceutical composition.

In another embodiment of the present disclosure, the excipient may comprise the binder, the disintegrant, the dispersant, the glidant, the lubricant and the solubilizer.

In the aforesaid embodiments, the binder may be hydroxypropylcellulose, microcrystalline cellulose or a combination thereof.

In the aforesaid embodiments, the disintegrant may be croscarmellose sodium.

In the aforesaid embodiments, the dispersant may be hypromellose acetate succinate.

In the aforesaid embodiments, the glidant may be silicon dioxide.

In the aforesaid embodiments, the lubricant may be sodium stearyl fumarate.

In the aforesaid embodiments, the solubilizer may be Macrogol 15 Hydroxystearate.

In the aforesaid embodiments, a content of the binder may be ranged from 5 wt % to 50 wt % based on a total weight of the pharmaceutical composition. For example, the content may be ranged from 10 wt % to 50 wt %, 15 wt % to 50 wt %, 20 wt % to 50 wt %, 20 wt % to 45 wt %, 25 wt % to 45 wt %, 25 wt % to 40 wt %, 25 wt % to 35 wt %, 25 wt % to 33 wt % or 28 wt % to 33 wt % based on a total weight of the pharmaceutical composition.

In the aforesaid embodiments, a content of the disintegrant may be ranged from 0.5 wt % to 10 wt % based on a total weight of the pharmaceutical composition. For example, the content may be ranged from 0.5 wt % to 8 wt %, 1 wt % to 8 wt %, 1 wt % to 7 wt %, 1 wt % to 6 wt %, 1 wt % to 5 wt %, 1.5 wt % to 5 wt %, 1.5 wt % to 4 wt %, 2 wt % to 4 wt %, 2 wt % to 3.5 wt % or 2.5 wt % to 3.5 wt % based on a total weight of the pharmaceutical composition.

In the aforesaid embodiments, a content of the dispersant may be ranged from 30 wt % to 70 wt % based on a total weight of the pharmaceutical composition. For example, the content may be ranged from 35 wt % to 70 wt %, 35 wt % to 65 wt %, 40 wt % to 65 wt %, 40 wt % to 60 wt %, 45 wt % to 60 wt %, 45 wt % to 55 wt % or 45 wt % to 50 wt % based on a total weight of the pharmaceutical composition.

In the aforesaid embodiments, a content of the glidant may be ranged from 0.5 wt % to 10 wt % based on a total weight of the pharmaceutical composition. For example, the content may be range from 0.5 wt % to 8 wt %, 0.5 wt % to 6 wt %, 0.5 wt % to 4 wt %, 0.8 wt % to 4 wt %, 0.8 wt % to 3 wt %, 0.8 wt % to 2 wt % or 0.8 wt % to 1.5 wt % based on a total weight of the pharmaceutical composition.

In the aforesaid embodiments, a content of the lubricant may be ranged from 0.5 wt % to 10 wt % based on a total weight of the pharmaceutical composition. For example, the content may be ranged from 0.5 wt % to 8 wt %, 0.5 wt % to 6 wt %, 0.5 wt % to 4 wt %, 1 wt % to 4 wt %, 1 wt % to 3 wt %, 1 wt % to 2.5 wt % or 1 wt % to 2 wt % based on a total weight of the pharmaceutical composition.

In the aforesaid embodiments, a content of the solubilizer is ranged from 1 wt % to 10 wt % based on a total weight of the pharmaceutical composition. For example, the content may be ranged from 1 wt % to 8 wt %, 2 wt % to 8 wt %, 2 wt % to 7 wt %, 3 wt % to 7 wt %, 3 wt % to 6 wt %, 4 wt % to 6 wt % or 5 wt % to 6 wt % based on a total weight of the pharmaceutical composition.

In another embodiment of the present disclosure, the excipient may comprise the binder, the disintegrant, the dispersant, the filler, the glidant, the lubricant and the solubilizer.

In the aforesaid embodiments, the binder may be hydroxypropylcellulose, microcrystalline cellulose or a combination thereof.

In the aforesaid embodiments, the disintegrant may be croscarmellose sodium.

In the aforesaid embodiments, the dispersant may be hypromellose acetate succinate.

In the aforesaid embodiments, the filler may be mannitol.

In the aforesaid embodiments, the glidant may be silicon dioxide. In the aforesaid embodiments, the lubricant may be sodium stearyl fumarate.

In the aforesaid embodiments, the solubilizer may be Macrogol 15 Hydroxystearate.

In the aforesaid embodiments, a content of the binder may be ranged from 1 wt % to 40 wt % based on a total weight of the pharmaceutical composition. For example, the content may be range from 1 wt % to 35 wt %, 5 wt % to 35 wt %, 10 wt % to 35 wt %, 15 wt % to 35 wt %, 15 wt % to 30 wt %, 18 wt % to 30 wt %, 18 wt % to 25 wt % or 18 wt % to 22 wt % based on a total weight of the pharmaceutical composition.

In the aforesaid embodiments, a content of the disintegrant may be ranged from 5 wt % to 15 wt % based on a total weight of the pharmaceutical composition. For example, the content may be ranged from 5 wt % to 13 wt %, 6 wt % to 13 wt %, 6 wt % to 12 wt %, 6 wt % to 11 wt %, 6 wt % to 10 wt %, 6 wt % to 9 wt % or 6 wt % to 8 wt % based on a total weight of the pharmaceutical composition.

In the aforesaid embodiments, a content of the dispersant may be ranged from 30 wt % to 70 wt %. For example, the content may be ranged from 35 wt % to 70 wt %, 35 wt % to 65 wt %, 40 wt % to 65 wt %, 40 wt % to 60 wt %, 43 wt % to 60 wt %, 43 wt % to 55 wt % or 43 wt % to 50 wt % based on a total weight of the pharmaceutical composition.

In the aforesaid embodiments, a content of the filler may be ranged from 3 wt % to 15 wt % based on a total weight of the pharmaceutical composition. For example, the content may be ranged from 3 wt % to 13 wt %, 5 wt % to 13 wt %, 6 wt % to 13 wt %, 6 wt % to 12 wt %, 6 wt % to 11 wt %, 6 wt % to 10 wt %, 6 wt % to 9 wt % or 6 wt % to 8 wt % based on a total weight of the pharmaceutical composition.

In the aforesaid embodiments, a content of the glidant may be ranged from 0.5 wt % to 10 wt % based on a total weight of the pharmaceutical composition. For example, the content may be range from 0.5 wt % to 8 wt %, 0.5 wt % to 6 wt %, 0.5 wt % to 4 wt %, 0.6 wt % to 4 wt %, 0.6 wt % to 3 wt %, 0.6 wt % to 2 wt %, 0.6 wt % to 1.5 wt % or 0.6% to 1 wt % based on a total weight of the pharmaceutical composition.

In the aforesaid embodiments, a content of the lubricant may be ranged from 0.5 wt % to 10 wt % based on a total weight of the pharmaceutical composition. For example, the content may be ranged from 0.5 wt % to 8 wt %, 0.5 wt % to 6 wt %, 0.5 wt % to 4 wt %, 1 wt % to 4 wt %, 1 wt % to 3 wt %, 1 wt % to 2.5 wt % or 1 wt % to 2 wt % based on a total weight of the pharmaceutical composition.

In the aforesaid embodiments, a content of the solubilizer may be ranged from 1 wt % to 10 wt % based on a total weight of the pharmaceutical composition. For example, the content may be ranged from 1 wt % to 8 wt %, 2 wt % to 8 wt %, 2 wt % to 7 wt %, 3 wt % to 7 wt %, 3 wt % to 6 wt %, 4 wt % to 6 wt % or 5 wt % to 6 wt % based on a total weight of the pharmaceutical composition.

In the aforesaid embodiments, the pharmaceutical composition comprises an effective amount of the active ingredient. The effective amount may be, for example, in a range from 0.01 to 2 mg per kg of the subject in need thereof.

In the aforesaid embodiments, the pharmaceutical composition can be formulated into powders. Herein, the method for preparing the powders of the pharmaceutical composition may be a spray drying method, but the present disclosure is not limited thereto.

When the powders of the pharmaceutical composition are prepared by the spray drying method, the inlet temperature may be ranged from 69° C. to 101° C. or 70° C. to 85° C. The outlet temperature may be ranged from 45° C. to 52° C. The atomization pressure may be ranged from 0.15 MPa to 0.22 Mpa. The blower may be ranged from scale 5 to scale 7. The air volume may be set at a range from 0.20 m³/min to 0.6 m³/min, for example 0.40 m³/min. The oxygen content may be set at <3%. However, the present disclosure is not limited thereto. The parameters for the spray drying method can be varied according to the diameters of the desired powders or the components of the pharmaceutical compositions.

In the present disclosure, the water poorly soluble active ingredient (ALP001E) can be prepared by the solvent spray drying method when the active ingredient is formulated into the pharmaceutical composition of the present disclosure. The powders of the pharmaceutical composition of the present disclosure show improved solubility or increased bioavailability. In addition, the specific formulation and process parameter of the pharmaceutical composition of the present disclosure can be implemented for pharmaceutical technology with process and operation advantages. For example, the inlet temperature with a gentle range from 69° C. to 101° C. can rapid produce spray dry powders and be easy to operate.

In the aforesaid embodiments, the powders of the pharmaceutical composition can be placed inside a capsule. The material of the capsule can be, for example, gelatin.

In the aforesaid embodiments, the powders of the pharmaceutical compostions can be compressed to form a tablet or a granule. When the pharmaceutical composition is formulated into the tablet or the particle, the pharmaceutical composition may further comprise a coating layer, wherein the active ingredient and the excipient are included within the coating layer.

In the aforesaid embodiments, the pharmaceutical composition can be an oral pharmaceutical composition, which can be formulated into powders, capsules, tablets or granules.

In the aforesaid embodiments, the pharmaceutical composition can be used in reducing the glycemic level in a subject thereof.

In the aforesaid embodiments, the pharmaceutical composition can be used in treating disorders associated with glucagon.

Also within the present disclosure is a method for reducing the glycemic level in a subject, comprising administering to the subject in need thereof the aforesaid pharmaceutical composition of the present disclosure.

Further covered by the present disclosure is a method of treating disorders associated with glucagon, comprising administering to a subject in need thereof the aforesaid pharmaceutical composition of the present disclosure.

In the present disclosure, the aforesaid subject can be a subject may be mammal, for example, a human, a pig, a horse, a cow, a dog, a cat, a mouse or a rat.

In the present disclosure, the diseases, conditions or disorders associated with glucagon can be, for example, hyperglycemia, Type II diabetes, metabolic syndrome, impaired glucose tolerance, glucosuria, diabetic nephropathy, diabetic neuropathy, diabetic retinopathy, hyperinsulinemia, insulin resistance syndrome, cataracts, obesity, dyslididemia, hypertension and myocardial infarction. However, the present disclosure is not limited thereto, and the pharmaceutical composition of the present disclosure can be applied to any other diseases, conditions or disorders associated with the glucagon signaling pathway. In one aspect of the present disclosure, the diseases, conditions or disorders associated with glucagon may be hyperglycemia, Type II diabetes, impaired glucose tolerance, insulin resistance syndrome or obesity. In another aspect of the present disclosure, the diseases, conditions or disorders associated with glucagon may be Type II diabetes.

The following embodiments are made to clearly exhibit the above-mentioned and other technical contents, features and/or effects of the present disclosure. Through the exposition by means of the specific embodiments, people would further understand the technical means and effects the present disclosure adopts to achieve the above-indicated objectives. Moreover, as the contents disclosed herein should be readily understood and can be implemented by a person skilled in the art, all equivalent changes or modifications which do not depart from the concept of the present disclosure should be encompassed by the appended claims.

Moreover, in the present specification, a value may be interpreted to cover a range within ±10% of the value, and in particular, a range within ±5% of the value, except otherwise specified; a range may be interpreted to be composed of a plurality of subranges defined by a smaller endpoint, a smaller quartile, a median, a greater quartile, and a greater endpoint, except otherwise specified.

### EXAMPLE

Without further elaboration, it is believed that one skilled in the art can, based on the above description, utilize the present disclosure to its fullest extent. The following specific examples are therefore to be construed as merely illustrative, and not limitative of the remainder of the disclosure in any way whatsoever. All publications cited herein are incorporated by reference in their entirety.

The present disclosure is explained by the following embodiments, which are not used to limit the scope of the present disclosure. Unless specified otherwise, “%” used herein for indicating the amount of the contents or the objects in the following embodiments are weight percentage.

### Chemicals

ALP001E: The preparation of ALP001E can be referred to US 20190345118.

Acetone: It is used as a solvent.

Methanol: It is used as a solvent.

Ethyl alcohol (200 Proof): It is used as a solvent.

Anhydrous dibasic calcium phosphate (Fujicalin): It is used as a direct compression excipient or a diluent and has exceptional flow and compression characteristics, while maintaining the ability for rapid disintegration. It is also named as dicalcium phosphate and calcium hydrogen phosphate.

Dibutyl sebacate: It is used as a plasticizer.

Croscarnellose sodium (Disolcel): It is an internally cross-linked sodium carboxymethylcellulose, which is used as a disintegrant in pharmaceutical formulations.

Hypromellose Acetate Succinate: It is used as a dispersant or as a carrier in a solid dispersion of ALP001E.

Hyfypromellose USP 2208 (Metolose 90SH 100SR or Metolose 90SH 4000SR): It is also named as hydroxypropylcellulose and is used as a binder for solid dosage forms such as tablets and granules. It also serves a variety of functions, for examples, in enhancing water retention, thickening, and acting as a protective colloid due to its surface activity, sustaining release, and film formation.

Soluplus: It is used as a binder and to achieve the solubilization effects in parallel, which is a polyvinyl caprolactam-polyvinyl acetate-polyethylene glycol graft copolymer.

Microcrystalline cellulose: It is widely used in pharmaceuticals as a binder/diluent in tablets and capsules due to its lubricant and disintegrant properties.

Eudragit ES100: It is used as a binder, which is a (meth)acrylic copolymer.

Macrogol 15 Hydroxystearate: it not only improves the drug solubility and absorption, but also is associated with product stability. It is a mixture of mainly monoesters and diesters of 12-hydroxystearic acid and macrogols obtained by the ethoxylation of 12-hydroxystearic acid. It is also known as 12-hydroxyoctadecanoic acid polymer with α-hydro-Ω-hydroxypoly(oxy-1,2-ethanediyl); 12-hydroxystearic acid polyethylene glycol copolymer; macrogol 15 hydroxystearate; polyethylene glycol-15-hydroxystearate; and polyethylene glycol 660 12-hydroxystearate.

Mannitol (Parteck M100 or M200): It is used as fillers.

Silicon dioxide (Syloid 244 FP): It is used as a glidant in the excipient part of the formulation. It has the capacity to take up excessive moisture from the other excipients while maintaining plasticizing properties.

Sodium stearyl fumarate (Lubripharm): It is an inert, hydrophilic, tablet lubricant and is useful in situations where other lubricating agents (i.e., magnesium stearate) fail to provide tablets of adequate stability, hardness, content uniformity, disintegration and dissolution rate.

Opadry 03K19229: It is used as a coating agent, and is a composition containing hydroxypropylmethyl cellulose, triacetin and tale.

Acryl-EZE 1 4932190002: It is an aqueous acrylic enteric coating system.

### Example 1

### <Preparation of Formulations A to F>

The components of Formulations A to F are listed in the following Table 1. The parameters of the manufacture process of spray drying for Formulations A to F are listed in the following Table 2.

The steps for preparing the spray dry powders of Formulations A to F are briefly described below.

Step 1: Dissolving hypromellose acetate succinate into acetone and stirring the solution until hypromellose acetate succinate was completely dissolved as a clear solution. The amounts of hypromellose acetate succinate and acetone are listed in Table 1.

Step 2: Dissolving Macrogol 15 Hydroxystearate into Ethyl Alcohol 200 Proof The solution B was prepared while Macrogol 15 Hydroxystearate was completely dissolved into EtOH. The recipe of the solution B is also listed in Table 1.

Step 3: Rapidly dissolving ALP001E into the clear solution prepared in Step 1 and stirring ALP001E and the solution until presenting as a clear solution (solution A).

Step 4: Mixing the solution A and the solution B in a beaker and keeping stirring in the blender.

Step 5: Injecting the mixed solution into the spray dryer. The parameters of spray dryer are described as Table 2 and the feeding gas was nitrogen. The inlet temperature was adjusted in the range from 70 to 85° C. until the outlet temperature was in the range from 42 to 52° C.

Step 6: Placing collected spray dry powders into the drying box for overnight (18-24 hr). This process is to remove the residue solvents to lower than 5000 ppm. The final product was stored in a glass bottle and kept in a refrigerator at −30° C. ALP001E and the final products may be unstable under high moisture condition. Therefore, the protocol should be executed in moisture control environment.

TABLE 1

| | Formulation A | | Formulation B | |
|---|---|---|---|---|
| Component | Amount | Percentage | Amount | Percentage |
| ALP001E | 10 mg | 22.4% | 10 mg | 18.3% |
| Hypromellose Acetate Succinate | 30 mg | 67.3% | 40 mg | 73.3% |
| Macrogol 15 Hydroxystearate | 4.6 mg | 10.3% | 4.6 mg | 8.4% |
| | (4466 μL) | | (4466 μL) | |
| Total | 44.6mg | 100% | 54.6mg | 100% |
| Acetone | 1600 mg | | 800 mg | |
| | (2030 mL) | | (1010 mL) | |
| Ethyl Alcohol 200 Proof | 20 mg | | 20 mg | |
| | (25.1 mL) | | (25.1 ml) | |
| | Formulation C | | Formulation D | |
| Component | Amount | Percentage | Amount | Percentage |
| ALPOOIE | 10 mg | 26.3% | 10 mg | 30.1% |
| Hypromellose Acetate Succinate | 23.4 mg | 61.6% | 18.6 mg | 56.0% |
| Macrogol 15 Hydroxystearate | 4.6 mg | 12.1% | 4.6 mg | 13.9% |
| | (4466 μL) | | (4466 μL) | |
| Total | 38 mg | 100% | 33.2 mg | 100% |
| Acetone | 1340 mg | — | 1150 mg | — |
| | (1700 ml) | | (1460 mL) | |
| Ethyl Alcohol 200 Proof | 20 mg | — | 20 mg | — |
| | (25.1 ml) | | (25.1 ml) | |
| | Formulation E | | Formulation F | |
| Component | Amount | Percentage | Amount | Percentage |
| ALP001E | 10.0 mg | 158% | 10.0 mg | 9.6% |
| Hypromellose Acetate Succinate | 48.8 mg | 77.0% | 90.0 mg | 86.0% |
| Macrogol 15 Hydroxystearate | 4.6 mg | 7.3% | 4.6 mg | 4.4% |
| | (4466 μL) | | (4466 μL) | |
| Total | 63.4 mg | 100% | 104.6 mg | 100% |
| Acetone* | 1306.0 mg | — | 2220.0 mg | — |
| | (1655 mL) | | (2814 mL) | |
| Ethyl Alcohol 200 Proof* | 20.0 mg | — | 20.0 mg | — |
| | (25.1 ml) | | (25.1 mL) | |

*To be evaporated during the drying process.

TABLE 2

| | Formulation A | Formulation B | Formulation C | Formulation D |
|---|---|---|---|---|
| Spray nozzle orifice size | 1A (Standard) (Particle size: 1 ~40 μm) | | | |
| Inlet temperature | 70~85° C. | 70-85° C. | 70~85° C. | 70~85° C. |
| Outlet temperature | 42~52° C. | 42~52° C. | 42~52° C. | 42~52° C. |
| Pump speed of solution | 3~4 (3.5) | 3~4 (3.5) | 3 | 3 |
| Atomization pressure | 0.20~0.22 Mpa | 0.15~0.20 Mpa | 0.20~0.22 Mpa | 0.20~0.22 Mpa |
| Blower | Scale 7 | Scale 7 | Scale 5 | Scale 5 |
| Air volume | 0.40 m³/min | 0.40 m³/min | 0.40 m³/min | 0.40 m³/min |
| Oxygen content (%) | <3% | <3% | <3% | <3% |

<Property measurement of Formulations A to D>

The properties of the obtained powders of Formulations A to D were measured, and the results are listed in the following Table 3. The measurement methods are briefly described below.

In the present example, all of the ingredients of the formulations were measured by the use of the electronic balance (M Mettler-Toledo Basic Moisture Analyzer) and converted to yield by calculation. The appearances of the formulations were check by the visual inspection.

To measure the degradation products from the formulations were using a HPLC system. The HPLC analysis was performed using an Agilent 1100 HPLC system comprising a quaternary pump, an in-line degasser, an auto-sampler and a photodiode array detector. The column employed was a Thermo Scientific Hypersil BDS C18 reverse phase column, 250 mm×4.6 mm, 5 μm.

A Metrohm Coulometer model 917 with Compact oven SC 885 auto sampler was used for the analysis. 50 mg of the powder sample was tested in triplicate, and the oven temperature was set to 105° C. The reagent used for analysis was hydranal Coulomat AG Oven.

Water content was also analysed using an AND MX-50 moisture balance. Approximately 1 g of material was placed on a metal pan and heated to 105° C. until the weight change is less than 0.01%.

DSC analysis was undertaken using a TA Instruments Q20 MDSC with auto sampler and refrigerated cooling accessory. Approximately 1-5 mg of sample was sealed in a TZero aluminium pan using a TZero pan press, To measure the mean particle size are using a powder particle size distribution instrument (Syepatec HELOS particle size analyzer).

In vitro dissolution testing was performed using an USP dissolution apparatus Type 2 (paddles) at 75 rpm (250 rpm at infinity) using 900 mL phosphate buffer pH 6.8 with 0.25% w/v SLS as the dissolution medium at 37±0.5° C.

TABLE 3

| | Formulation A | Formulation B | Formulation C | Formulation D |
|---|---|---|---|---|
| Yield (%) | 50~75% | 75~95% | 50~75% | 50~75% |
| Appearance | No aggregation | No aggregation | Aggregation, poor | Aggregation, poor |
| | | | dispersion | dispersion |
| Degradation | individual | individual | individual | individual |
| products | degradation | degradation | degradation | degradation |
| (HPLC)¹ | NMT <0.1% | NMT <0.1% | NMT <0.1% | NMT <0.1% |
| Water content | <6% | <3% | <6% | <6% |
| (%) | | | | |
| DSC¹ | Amorphous | Amorphous | Tₘ peak of | Tₘ peak of |
| | | | ALP001E peak | ALP001E peak |
| Mean particle size (μm) | 8.02 | 15.22 | 30.41 | ND |

Solubility² (fold)

| | | | | |
|---|---|---|---|---|
| USP, pH 6.8 | 120.23 | 107.27 | 30.91 | ND |

¹The dry powders of Formulations A to D were placed at 60° C. for 7 days, and these data indicated the thermal stability of Formulations A to D.
²The solubility of the dry powders of Formulations A to D was meansured at the condition of USP, pH 6.8 and compared with the solubility of APL001E (0.00044 mg/mL).

The appearance, the impurity and the thermal stability of the dry powders of Formulations A to D were measured by placing the dry powders of Formulations A to D at 60° C. for 7 days (Day 7) and compared with the fresh dry powders of Formulations A to D (Day 0). From the results shown in Table 3, no significant appearance changes were observed in the dry powders of Formulations A and B at Day 7 compared with the dry powders of Formulations A and B at Day 0. In addition, the HPLC data indicate that no more than 0.1% impurity was observed in the dry powders of Formulas A to D at Day 7. Furthermore, the DSC data of the dry powders of Formulation A and B show no significant change at Day 7 compared to the dry powders of Formulation A and B at Day 0, but Tₘ peak of ALP001E peak (Tₘ range: from 120° C. to 125° C.) between 115° C. and 130° C. was observed in the dry powders of Formulations C and D at Day 7 compared to the dry powders of Formulations C and D at Day 0. These results indicate the dry powders of Formulations A to D have good stability, and in particular, the dry powders of Formulations A and B have excellent stability.

In addition, the solubility of the dry powders of Formulations A and B are more than 100 folds compared with the solubility of APL001E, and these data indicate that the dry powders of Formulations A and B have improved solubility.

Furthermore, as shown in Table 3, the ranges of particle distributions of the dry powders of Formulations A to C are not the same. In the dry powders of Fomulation A, D10 (Dv 0.1) is 1˜3 μm, D50 (Dv 0.5) is 4˜10 μm, and D90 (Dv 0.9) is 11˜20 μm. In the dry powders of Formulation B, D10 (Dv 0.1) is 1˜3 μm., D50 (Dv 0.5) is 5˜15 μm, and D90 (Dv 0.9) is 25˜50 μm. In the dry powders of Formulation C, D10 (Dv 0.1) is 3˜6 μm, D50 (Dv 0.5) is 20˜35 μm, and D90 (Dv 0.9) is 50˜70 μm. Herein, D50 (Dv 0.5) is the particle size of the median for a volume distribution; and D10 (Dv 0.1) and D90 (Dv 0.9) respectively the particle size below which 10% and 90% of the volume of particles exists.

<Oral bioavailability of Formulations A to C>

The pharmacokinetics (PK) testing method for oral bioavailability is described below.

Briefly, three healthy, research Beagle dogs, aged 4˜10 years and body weight 6˜11 kg were used. The pharmacokinetics experiments have intravenous bolus (IV bolus) and oral groups (each n=3).

For the IV bolus group, ALP001. (an active metabolite compound from ALP001E, represented by the following formula (II)) dissolved in vehicle (0.1M NaHCO₃, pH7.4) was administered to the dogs via IV bolus with a dose strength ranging from 0.01 mg/kg to 2.00 mg/kg depending on the experiment design.

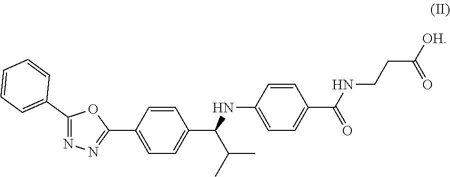 [SMILES: CC(C)[C@H](Nc1ccc(C(=O)NCCC(=O)O)cc1)c1ccc(-c2nnc(-c3ccccc3)o2)cc1]

For the oral group, the capsule comprising dry powders of Formulations A, B or C was orally administered to the dogs. The dogs were weighted and the blood of the dogs was taken before the capsule was administered. The capsule comprising dry powders Formulations A, B or C was stuffed into the dog's mouth (wherein the dose of the active ingredient ALP001E is ranged from 0.01 mg/kg to 2.00 mg/kg, depending on experiment design) and 3 ml water was given immediately after the capsule comprising the dry powders of Formulations A, B or C was stuffed into the dog's mouth.

For the IV bolus and oral groups, the dogs received the drugs after fasting for 4 hours, and the blood samples of the dogs were taken before administration. The blood samples were collected at pre-assigned times (10 min, 1 hr, 2 hr, 4 hr, 5 hr, 7 hr and 24 hr) after administration. The concentrations of the drug in plasma were determined with validated high performance liquid chromatography.

The plasma concentration data were analyzed and modelled by the non-compartmental method to obtain pharmacokinetics parameters, such as AUC (area under the curve) and Cₘₐₓ (maximum concentration observed). In addition, the bioavailability was obtained by comparing the data of the oral group with the data of the IV bolus group. The results are listed in the following Table 4.

TABLE 4

| | Oral AUCₐₗₗ | IV AUCₐₗₗ | Bioavailability |
|---|---|---|---|
| Formulation | (μg.h/ml) | (μg.h/m) | (%) |
| A | 0.78 | 2.18 | 35.8% |
| B | 1.27 | 2.18 | 58.4% |
| C | 0.54 | 2.18 | 24.5% |

The results shown in Table 4 indicate that the oral dry powders of Formulations A to C, in particular the oral dry powders of Formulation B, have good bioavailability. In addition, the oral bioavailability of dry powders is increased as the ratio of hypromellose acetate succinate to Macrogol 15 Hydroxystearate increased. In particular, the oral bioavailability of the dry powders of Formulation B is 58.4%, which is the best in all formulation in dog PK study.

### Example 2

### <Preparation of Formulations G and H>

The components of Formulations U and H are respectively listed in the following Tables 5 and 6. The parameters of the manufacture process of spray drying for Formulations G and H are listed in the following Table 7.

The steps for preparing the tablets of Formulation G are briefly described below.

Step 1: Dissolving Hypromellose Acetate Succinate, Dibutyl Sebacate and ALP001F in acetone and stirring the solution until completely dissolved into a clear solution.

Step 2: injecting the mixed solution into the spray dryer. The parameters of spray dryer are described as Table 7 and the feeding gas was nitrogen. The inlet temperature was adjusted to 85° C. until the outlet temperature was 41° C.

Step 3: Placing collected spray dry powder into the drying box for overnight (18˜24 hr). This process is to remove the residue solvents to lower than 5000 ppm. The final product was stored in a glass bottle and kept in a refrigerator at −30° C.

Step 4: The spray dry powders (45.10 parts by weight) was blended in a blender with anhydrous dibasic calcium phosphate (16.30 parts by weight), microcrystalline cellulose (32.50 parts by weight), croscarmellose sodium (3.00 parts by weight), silicon dioxide (2.00 parts by weight) and sodium stearyl fumarate (1.30 parts by weight).

Step 5: The powder blend was compressed to tablets of 40.0 mg on a single punch tablet press.

Step 6: The tablets were sub-coated in a coating pan by spraying an aqueous dispersion for film coating at a temperature of 40° C.

Step 7: The sub-coated tablets were then enteric coated in a coating pan by spraying an aqueous dispersion for enteric coating at a temperature of 32° C.

TABLE 5
Formulation G

| | | |
|---|---|---|
| Component | Amount (mg) | Percent |

Core

| | | |
|---|---|---|
| ALP001E | 5.00 | 12.50% |
| Hypromellose Acetate | 11.70 | 29.30% |
| Succinate | | |
| Dibutyl Sebacate (DBS) | 1.30 | 3.30% |
| Anhydrous Dibasic Calcium | 6.50 | 16.30% |

Phosphate

| | | |
|---|---|---|
| Microcrystalline Cellulose | 13.00 | 32.50% |
| Croscarmellose Sodium | 1.20 | 3.00% |
| Silicon Dioxide | 0.80 | 2.00% |
| Sodium Stearyl Fumarate | 0.50 | 1.30% |
| Total | 40.00 | 100% |
| Acetone* | 175.0 mg | — |
| | (222 μL) | |

Subcoating

| | | |
|---|---|---|
| Opadry 03K19229 | 0.8 | 100% |

Enteric Coating

| | | |
|---|---|---|
| Acryl-EZE II 493Z190002 | 4.08 | 100% |

*To be evaporated during the drying process.

The steps for preparing the tablets of Formulation H are briefly described below.

Step 1: Dissolving hypromellose acetate succinate into acetone and stirring the solution until hypromellose acetate succinate was completely dissolved as clear solution.

Step 2: Dissolving Macrogol 15 Hydroxystearate into Ethyl Alcohol 200 Proof. The solution B was prepared while Macrogol 15 Hydroxystearate was completely dissolved into EtOH.

Step 3: Rapidly dissolving ALP001E into the clear solution prepared in the step 1 and stirring ALP01GE and the solution until presenting as a clear solution (solution A).

Step 4: Mixing the solution A and the solution B in a beaker and keeping stirring in the blender.

Step 5: Injecting the mixed solution into the spray dryer. The parameters of spray dryer are described as Table 7 and the feeding gas was nitrogen. The inlet temperature was adjusted to 85° C. until the outlet temperature was 41°.

Step 6: Placing collected spray dry powders into the drying box for overnight (18˜24 hr). This process is to remove the residue solvents to lower than 5000 ppm. The final product was stored in a glass bottle and kept in a refrigerator at −30° C.

Step 7: The spray dried powders (54.60 parts by weight) was blended in a blender with metolose 90SH 100SR (16.00 parts by weight), metolose 90SH 4000SR (16.00 parts by weight), anhydrous dibasic calcium phosphate (10.00 parts by weight), silicon dioxide (2.00 part by weight) and sodium stearyl fumarate (1.40 parts by weight).

Step 8: The powdery blend was compressed to tablets on a single punch tablet press, Step 9: The tablets were then sub-coated in a coating pan by spraying an aqueous dispersion for film coating at a temperature of 40° C.

Step 10: The sub-coated tablets were then enteric coated in a coating pan by spraying an aqueous dispersion for enteric coating at a temperature of 32° C.,

TABLE 6
Formulation H

| | | |
|---|---|---|
| Component | Amount (mg) | Percent |

Core

| | | |
|---|---|---|
| ALP001E | 5 | 10% |
| Hypromellose Acetate | 20 | 40.00% |
| Succinate | | |
| Macrogol 15 Hydroxystearate | 2.3 | 4.60% |
| Metolose 90SH 100SR | 8 | 16.00% |
| Metolose 90SH 4000SR | 8 | 16.00% |
| Anhydrous Dibasic Calcium | | 10.00% |
| Phosphate | | |
| Silicon Dioxide | 1 | 2.00% |
| Sodium Stearyl Fumarate | 0.7 | 1.40% |
| Total | 50 | 100% |
| Acetone* | 555.6 mg | — |
| | (704 μL) | |
| Ethyl Alcohol 200 Proof* | 10.0 mg | — |
| | (12.6 μL) | |

Subcoating

| | | |
|---|---|---|
| Opadry 03K19229 Clear | 1 | 100% |

Enteric Coating

| | | |
|---|---|---|
| Acryl-EZE II 493Z190002 | 5.1 | 100% |
| Clear | | |

*To be evaporated during the drying process.

TABLE 7

| Process parameter | Range of value |
|---|---|
| Spray nozzle orifice size | 1A (Standard) (Particle size: 1~40 μm) |
| Inlet temperature | 85° C. |
| Outlet Temperature | 41° C. |
| Pump speed of solution | 3 |
| Atomization pressure | 0.06 Mpa |
| Blower | start setting (scale 6), end setting (scale 9) |
| Air volume | 0.40 m³/min |
| Oxygen content (%) | <3% |

### Example 3

### <Preparation of Formulations 1 and J>

The components of Formulations I and J are respectively listed in the following Tables 8 and 9. The parameters of the manufacture process of spray drying for Formulations I and J are listed in the following Table 10.

The steps for preparing the spray dry powders of Formulation I are briefly described below.

Step 1: Dissolving Eudragit FS100 into acetone and stirring the solution until Eudragit FS100 was completely dissolved as a clear solution. The amounts of Eudragit FS100 and acetone are listed in Table 8.

Step 2: Dissolving Macrogol 15 Hydroxystearate into Ethyl Alcohol 200 Proof. The solution 3 was prepared while Macrogol 15 Hydroxystearate was completely dissolved into EtOH. The recipe of the solution B is also listed in Table 8, Step 3: Rapidly dissolving ALP001E into the clear solution prepared in the step 1 and stirring ALP001E and the solution until presenting as a clear solution (solution A).

Step 4: Mixing the solution A and the solution B in a beaker and keeping stirring in the blender.

Step 5: Injecting the mixed solution into the spray dryer. The parameters of spray dryer are described as Table 10 and the feeding gas was nitrogen. The inlet temperature was adjusted to 100° C. until the outlet temperature was in the range from 41 to 47° C.

Step 6: Placing collected spray dry powders into the drying box for overnight (18˜24 hr). This process is to remove the residue solvents to lower than 5000 ppm. The final product was stored in a glass bottle and kept in a refrigerator at −30° C. ALP001E and the final product may be unstable under high moisture condition. Therefore, the protocol should be executed in moisture control environment.

TABLE 8
Formulation I

| Component | Amount (mg) | Percent |
|---|---|---|
| ALP001E | 5.00 | 18.32% |
| Eudragit FS100 | 20.00 | 73.26% |
| Macrogol 15 Hydroxystearate | 2.30 | 8.42% |
| Total | 27.30 | 100.00% |
| Acetone* | 400.00 | — |
| Ethyl Alcohol 200 Proof* | 5.00 | — |

*To be evaporated during the drying process.

The steps for preparing the spray dry powders of Formulation J are briefly described below.

Step 1: Dissolving Soluplus, Macrogol 15 hydroxystearate and ALP001E in methanol, and stirring the solution until completely dissolved into a clear solution. The amount of Soluplus and acetone is listed in Table 9.

Step 2: Injecting the mixed solution into the spray dryer. The parameters of spray dryer are described as Table 10 and the feeding gas was nitrogen. The inlet temperature was adjusted to 100° C. until outlet temperature was in the range from 41 to 47° C.

Step 3: Placing collected spray dry powders into the drying box for overnight (18˜24 hr). This process is to remove the residue solvents to lower than 5000 ppm. The final product was stored in a glass bottle and kept in a refrigerator at −30° C. ALP001E and the final product may be unstable under high moisture condition. Therefore, the protocol should be executed in moisture control environment.

TABLE 9
Formulation J

| Component | Amount (mg) | Percent |
|---|---|---|
| ALP001E | 5.00 | 18.32% |
| Soluplus | 20.00 | 73.26% |
| Macrogol 15 Hydroxystearate | 2.30 | 8.42% |
| Total | 27.3 | 100.00% |
| Methanol* | 400.0 mg | — |
| | (505 μL) | |

*To be evaporated during the drying process.

TABLE 10

| Process parameter | Range of value |
|---|---|
| Spray nozzle orifice size | 1A (Standard) (Particle size: 1~40 μm) |
| Inlet temperature | 100° C. |
| Outlet Temperature | 41-47° C. |
| Pump speed of solution | 3~6 (5) |
| Atomization pressure | 0.06 Mpa |
| Blower | start setting (scale 6), end setting (scale6) |
| Air volume | 0.40 m³/min |
| Oxygen content (%) | <3% |

### Example 4

### <Preparation of Formulation K>

The components of Formulation K. are respectively listed in the following Table 11 The parameters of the manufacture process of spray drying for Formulation K are listed in the following Table 12.

The steps for preparing the tablets of Formulation K are briefly described below.

Step 1: Dissolving Hypromellose Acetate Succinate into acetone and stirring the solution until Hypromellose Acetate Succinate was completely dissolved as a clear solution. The amounts of Hypromellose Acetate Succinate and acetone are listed in Table 11.

Step 2: Dissolving Macrogol 15 Hydroxystearate into Ethyl Alcohol 200 Proof The solution B was prepared while Macrogol 15 Hydroxystearate was completely dissolved into EtOH. The recipe of the solution B is also listed in Table 11.

Step 3: Rapidly dissolving ALP001 E into the clear solution prepared in the step 1 and stirring ALP001E and the solution until presenting as a clear solution (solution A).

Step 4: Mixing the solution A and the solution B in the beaker and keeping stirring in the blender.

Step 5: Injecting the mixed solution into the spray dryer. The parameters of spray dryer are described as Table 12 and the feeding gas was nitrogen. The inlet temperature was adjusted to 85° C. until the outlet temperature was in the range from 41 to 47° C.

Step 6: Placing collected spray dry powders into the drying box for overnight (18˜24 hr). This process is to remove the residue solvents to lower than 5000 ppm. The final product was stored in a glass bottle and kept in a refrigerator at −30° C. ALP001E and the final product may be unstable under high moisture condition. Therefore, the protocol should be executed in moisture control environment.

Step 7: The spray dried powder (64.99 parts by weight) was blended in a blender with hydroxypropylcellulose (5.21 parts by weight), microcrystalline cellulose (24.06 parts by weight), croscarmellose sodium (3.12 parts by weight), silicon dioxide (1.04 parts by weight) and sodium stearyl fumarate (1.58 parts by weight).

Step 8: The powdery blend was compressed to tablets of 336.0 mg on a single punch tablet press.

Step 9: The tablets were then sub-coated in a coating pan by spraying an aqueous dispersion for film coating at a temperature of 40° C.

Step 10: The sub-coated tablets were then enteric coated in a coating pan by spraying an aqueous dispersion for enteric coating at a temperature of 32° C.

TABLE 11
Formulation K

| | | |
|---|---|---|
| Component | Amount (mg) | Percent |

Core

| | | |
|---|---|---|
| ALP001E | 40 | 11.90% |
| Hypromellose Acetate Succinate | 160 | 47.61% |
| Macrogol 15 Hydroxystearate | 18.4 | 5.48% |
| Hydroxypropylcellulose | 17.5 | 5.21% |
| Microcrystalline Cellulose | 80.86 | 24.06% |
| Croscarmellose Sodium | 10.5 | 3.12% |
| Silicon Dioxide | 3.5 | 1.04% |
| Sodium Stearyl Fumarate | 5.3 | 1.58% |
| Total | 336.06 | 100.00% |
| Acetone* | 4444.4 mg | — |
| | (5633 μL) | |
| Ethyl Alcohol 200 Proof* | 80.0 mg | — |
| | (100.5 μL) | |

Subcoating

| | | |
|---|---|---|
| Opadry 03K19229 | 6.72 | 100.00% |

Enteric Coating

| | | |
|---|---|---|
| Acryl-EZE II 493Z190002 | 34.28 | 100.00% |

*To be evaporated during the drying process.

TABLE 12

| Process parameter | Range of value |
|---|---|
| Spray nozzle orifice size | IA (Standard) (Particle size: |
| | 1~40 μm) |
| Inlet temperature | 85° C. |
| Outlet Temperature | 41~47° C. |
| Pump speed of solution | 3~6 (5) |
| Atomization pressure | 0.06 Mpa |
| Blower | start setting (scale 6), |
| | end setting (scale 9) |
| Air volume | 0.40 m³/min |
| Oxygen content (%) | <3% |

### Example 5

### <Preparation of Formulation L>

The components of Formulation L are respectively listed in the following Table 13. The parameters of the manufacture process of spray drying for Formulation L are listed in the following Table 14.

The steps for preparing the tablets of Formulation L are briefly described below.

Step 1: Dissolving Hypromellose Acetate Succinate into acetone and stirring the solution until Hypromellose Acetate Succinate was completely dissolved as a clear solution. The amount w of 1-Hypromellose Acetate Succinate and acetone are listed in Table 13.

Step 2: Dissolving Macrogol 15 Hydroxystearate into Ethyl Alcohol 200 Proof. The solution B was prepared while Macrogol 15 Hydroxystearate was completely dissolved into EtOH, The recipe of the solution B is also fisted in Table 13.

Step 3: Rapidly dissolving ALP001E into the clear solution prepared in the step 1 and stirring ALP001E and the solution until presenting as a clear solution (solution A).

Step 4: Mixing the solution A and the solution B in a beaker and keeping stirring in the blender.

Step 5: Injecting the mixed solution into the spray dryer. The parameters of spray dryer are described as Table 14 and the feeding gas was nitrogen. The inlet temperature was adjusted to 85° C. until the outlet temperature was in the range from 41 to 47° C.

Step 6: Placing collected spray dry powders into the drying box for overnight (18˜24 hr), This process is to remove the residue solvents to lower than 5000 ppm. The final product was stored in a glass bottle and kept in a refrigerator at −30° C. ALP001E and the final product may be unstable under high moisture condition. Therefore, the protocol should be executed in moisture control environment.

Step 7: The spray dried powders (62.40 parts by weight) was blended in a blender with hydroxypropylcellulose (1.90 parts by weight), microcrystalline cellulose (19.90 parts by weight), mannitol (6.60 parts by weight), croscarmellose sodium (7.00 parts by weight), silicon dioxide (0.70 part by weight) and sodium stearyl fumarate (1.50 parts by weight).

Step 8: The powdery blend was compressed to tablets of 350.0 mg on a single punch tablet press.

Step 9: The tablets were then sub-coated in a coating pan by spraying an aqueous dispersion for film coating at a temperature of 40° C.

Step 10: The sub-coated tablets were then enteric coated in a coating pan by spraying an aqueous dispersion for enteric coating at a temperature of 32° C.

TABLE 13
Formulation L

| | | |
|---|---|---|
| Component | Amount (mg) | Percent |

Core

| | | |
|---|---|---|
| ALP001E | 40 | 11.40% |
| Hypromellose Acetate Succinate | 160 | 45.70% |
| Macrogol 15 Hydroxystearate | 18.4 | 5.30% |
| Hydroxypropylcellulose | 6.5 | 1.90% |
| Microcrystalline Cellulose | 69.6 | 19.90% |
| Mannitol | 23.2 | 6.60% |
| Croscarmellose Sodium | 24.5 | 7.00% |
| Silicon Dioxide | 2.5 | 0.70% |
| Sodium Stearyl Fumarate | 5.3 | 1.50% |
| Total | 350 | 100% |
| Acetone* | 4444.4 mg | — |
| | (5633 μL) | |
| Ethyl Alcohol 200 Proof* | 80.0 mg | — |
| | (100.5 μL) | |

Subcoating

| | | |
|---|---|---|
| Opadry 03K19229 | 7 | 100% |

Enteric Coating

| | | |
|---|---|---|
| Acryl-EZE II 4932190002 | 35.7 | 100% |

*To be evaporated during the drying process.

TABLE 14

| Process parameter | Range of value |
|---|---|
| Spray nozzle orifice size | 1A (Standard) (Particle size: 1~40 μm) |
| Inlet temperature | 85° C. |
| Outlet Temperature | 41~47° C. |
| Pump speed of solution | 3~6 (5) |
| Atomization pressure | 0.06 Mpa |
| Blower | setting (scale 8) |
| Air volume | 0.40 m³/min |
| Oxygen content (%) | <3% |

From the results shown above, the present disclosure provides a novel pharmaceutically composition, which has improved stability, acceptable solubility or increased oral absorption. Thus, the pharmaceutical composition of the present disclosure can be used for reducing the glycemic level or treating disorders associated with glucagon in a subject thereof by oral administration.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

Further, from the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

What is claimed is:

1. A pharmaceutical composition, comprising:
an active ingredient of the following formula (I) or a pharmaceutically acceptable salt or solvate thereof: 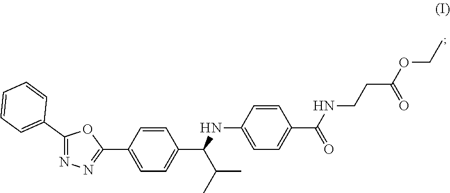 [SMILES: CCOC(=O)CCNC(=O)c1ccc(N[C@H](c2ccc(-c3nnc(-c4ccccc4)o3)cc2)C(C)C)cc1]
one or more excipient selected from the group consisting of a binder, a diluent, a disintegrant, a dispersant, an emulsifier, a filler, a glidant, a lubricant, a plasticizer, a solubilizer, a thickener and a wetting agent.

2. The pharmaceutical composition of claim 1, which is an oral pharmaceutical composition.

3. The pharmaceutical composition of claim 1, wherein a content of the active ingredient is ranged from 5 wt % to 50 wt % based on a total weight of the pharmaceutical compostions.

4. The pharmaceutical composition of claim 1, comprising the dispersant and the solubilizer.

5. The pharmaceutical composition of claim 4, wherein the dispersant is hypromellose acetate succinate.

6. The pharmaceutical composition of claim 4, wherein the solubilizer is Macrogol 15 Hydroxystearate.

7. The pharmaceutical composition of claim 5, wherein a content of the dispersant is ranged from 40 wt % to 90 wt % based on a total weight of the pharmaceutical composition.

8. The pharmaceutical composition of claim 6, wherein a content of the solubilizer is ranged from 3 wt % to 20 wt % based on a total weight of the pharmaceutical composition.

9. The pharmaceutical composition of claim 4, wherein a weight ratio of the dispersant to the solubilizer is ranged from 3.0 to 20.

10. The pharmaceutical composition of claim 1, comprising the binder, the disintegrant, the dispersant, a diluent, the glidant, the lubricant and the plasticizer.

11. The pharmaceutical composition of claim 10, wherein the binder is microcrystalline cellulose, the disintegrant is croscarmellose sodium, the dispersant is hypromellose acetate succinate, the diluent is dibasic calcium phosphate, the glidant is silicon dioxide, the lubricant is sodium stearyl fumarate and the plasticizer is dibutyl sebacate.

12. The pharmaceutical composition of claim 10, wherein a content of the binder is ranged from 15 wt % to 50 wt %, a content of the disintegrant is ranged from 0.5 wt % to 10 wt %, a content of the dispersant is ranged from 15 wt % to 50 wt %, a content of the diluent is ranged from 10 wt % to 30 wt %, a content of the glidant is ranged from 0.5 wt % to 10 wt %, a content of the lubricant is ranged from 0.5 wt % to 10 wt % and a content of the plasticizer is ranged from 0.5 wt % to 10 wt % based on a total weight of the pharmaceutical composition.

13. The pharmaceutical composition of claim 1, comprising the binder, the dispersant, the diluent, the glidant, the lubricant and the solubilizer.

14. The pharmaceutical composition of claim 13, wherein the binder is metolose, the dispersant is hypromellose acetate succinate, the diluent is dibasic calcium phosphate, the glidant is silicon dioxide, the lubricant is sodium stearyl fumarate and the solubilizer is Macrogol 15 Hydroxystearate.

15. The pharmaceutical composition of claim 13, wherein a content of the binder is ranged from 15 wt % to 50 wt %, a content of the the dispersant is ranged from 20 wt % to 60 wt %, a content of the diluent is ranged from 5 wt % to 20 wt %, a content of the glidant is ranged from 0.5 wt % to 10 wt %, a content of the the lubricant is ranged from 0.5 wt % to 10 wt % and a content of the the solubilizer is ranged from 1 wt % to 10 wt % based on a total weight of the pharmaceutical composition.

16. The pharmaceutical composition of claim 1, comprising the binder and the solubilizer.

17. The pharmaceutical composition of claim 16, wherein the binder is Eudragit FS100, soluplus or combination thereof, and the solubilizer is Macrogol 15 Hydroxystearate.

18. The pharmaceutical composition of claim 16, wherein a content of the binder is ranged from 40 wt % to 80 wt %, and a content of the solibilizer is 5 wt % to 15 wt % based on a total weight of the pharmaceutical composition.

19. The pharmaceutical composition of claim 1, comprising the binder, the disintegrant, the dispersant, the glidant, the lubricant and the solubilizer.

20. The pharmaceutical composition of claim 19, wherein the binder is hydroxypropylcellulose, microcrystalline cellulose or a combination thereof, the disintegrant is croscarmellose sodium, the dispersant is hypromellose acetate succinate, the glidant is silicon dioxide, the lubricant is sodium stearyl fumarate and the solubilizer is Macrogol 15 Hydroxystearate.

21. The pharmaceutical composition of claim 19, wherein a content of the binder is ranged from 5 wt % to 50 wt %, a content of the disintegrant is ranged from 0.5 wt % to 10 wt %, a content of the dispersant is ranged from 30 wt % to 70 wt %, a content of the glidant is ranged from 0.5 wt % to 10 wt %, a content of the lubricant is ranged from 0.5 wt % to 10 wt % and a content of the solubilizer is ranged from 1 wt % to 10 wt % based on a total weight of the pharmaceutical composition.

22. The pharmaceutical composition of claim 1, comprising the binder, the disintegrant, the dispersant, the tiller, the glidant, the lubricant and the solubilizer.

23. The pharmaceutical composition of claim 22, wherein the binder is hydroxypropylcellulose, microcrystalline cellulose or a combination thereof, the disintegrant is croscarmellose sodium, the dispersant is hypromellose acetate succinate, the filler is mannitol, the glidant is silicon dioxide, the lubricant is sodium stearyl fumarate and the solubilizer is Macrogol 15 Hydroxystearate.

24. The pharmaceutical composition of claim 22, wherein a content of the binder is ranged from 1 wt % to 40 wt %, a content of the disintegrant is ranged from 5 wt % to 15 wt %, a content of the dispersant is ranged from 30 wt % to 70 wt %, a content of the filler is ranged from 3 wt % to 15 wt %, a content of the glidant is ranged from 0.5 wt % to 10 wt %, a content of the lubricant is ranged from 0.5 wt % to 10 wt % and a content of the solubilizer is ranged from 1 wt % to 10 wt % based on a total weight of the pharmaceutical composition.

25. The pharmaceutical composition of claim 1, further comprising a coating layer, wherein the active ingredient and the excipient are included within the coating layer.

26. The pharmaceutical composition of claim 1 for used in reducing the glycemic level in a subject thereof.

27. The pharmaceutical composition of claim 1 for used in treating disorders associated with glucagon.

28. A method for reducing the glycemic level in a subject, comprising administering to the subject in need thereof a pharmaceutical composition, wherein the pharmaceutical composition comprises:
an active ingredient of the following formula (I) or a pharmaceutically acceptable salt or solvate thereof: 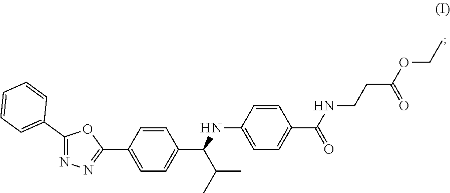 [SMILES: CCOC(=O)CCNC(=O)c1ccc(N[C@H](c2ccc(-c3nnc(-c4ccccc4)o3)cc2)C(C)C)cc1]
and
one or more excipient selected from the group consisting of a binder, a diluent, a disintegrant, a dispersant, an emulsifier, a filler, a glidant, a lubricant, a plasticizer, a solubilizer, a thickener and a wetting agent.

29. A method of treating disorders associated with glucagon, comprising administering to a subject in need thereof a pharmaceutical composition, wherein the pharmaceutical composition comprises:
an active ingredient of the following formula (I) or a pharmaceutically acceptable salt or solvate thereof: 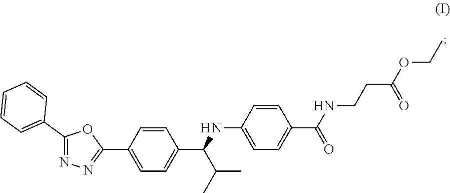 [SMILES: CCOC(=O)CCNC(=O)c1ccc(N[C@H](c2ccc(-c3nnc(-c4ccccc4)o3)cc2)C(C)C)cc1]
and
one or more excipient selected from the group consisting of a binder, a diluent, a disintegrant, a dispersant, an emulsifier, a filler, a glidant, a lubricant, a plasticizer, a solubilizer, a thickener and a wetting agent.
